(19) United States
(12) Patent Application Publication
(10) Pub. No.: US 2022/0280124 A1
(43) Pub. Date: Sep. 8, 2022

(54) DIAGNOSIS SUPPORT SYSTEM

(71) Applicant: Canon Medical Systems Corporation, Otawara-shi (JP)

(72) Inventors: Atsuko SUGIYAMA, Nasushiobara (JP); Joanne Désirée Schuijf, Leiden (NL); Jacqueline Hislop-Jambrich, Victoria (AU)

(73) Assignee: Canon Medical Systems Corporation, Otawara-shi (JP)

(21) Appl. No.: 17/653,713

(22) Filed: Mar. 7, 2022

(30) Foreign Application Priority Data
Mar. 8, 2021 (JP) 2021-036566

(51) Int. Cl. A61B 6/03; G06T 7/00; A61B 6/00

(52) U.S. Cl. CPC A61B 6/032; G06T 7/0012; A61B 6/503; G06T 2207/10081; G06T 2207/30048

(57) ABSTRACT

According to one embodiment, a diagnosis support system evaluates a cardiovascular disease risk by using extra-coronary calcification as a trigger. The diagnosis support system includes a processing circuit. The processing circuit is configured to acquire image interpretation information related to an image interpretation result of a photographed image of a patient, determine whether calcification is present or not based on the image interpretation information, evaluate the cardiovascular disease risk based on medical information on a patient, and output a determination result and an evaluation result.

### CROSS-REFERENCE TO RELATED APPLICATIONS

This application is based upon and claims the benefit of priority from Japanese Patent Application No. 2021-036566, filed Mar. 8, 2021, the entire contents of which are incorporated herein by reference.

### FIELD

Embodiments described herein relate generally to a diagnosis support system.

### BACKGROUND

As a method of evaluating a risk of developing cardiovascular disease (CVD) (hereinafter referred to as a cardiovascular disease risk), a method of calculating a numerical value (hereinafter referred to as a CVD risk value) indicating the cardiovascular disease risk by using age, gender, lifestyle, various medical examination results, etc., is known.

It is necessary to detect a patient at a high cardiovascular disease risk at an early stage, recommend prevention of cardiovascular disease, and reduce the cardiovascular disease risk.

### BRIEF DESCRIPTION OF THE DRAWINGS

FIG. 1 is a diagram showing an example of a configuration of a diagnosis support system according to a first embodiment.

FIG. 2 is a diagram showing an example of breast artery calcification detected based on a mammography image.

FIG. 3 is a diagram showing an example of aorta calcification detected based on a chest X-ray image.

FIG. 4 is a diagram showing an example of aorta calcification detected based on a CT image of the lung field in an axial cross section.

FIG. 5 is a diagram showing an example of aorta calcification detected based on a CT image of the lung field in a sagittal cross section.

FIG. 6 is a diagram showing an example of coronary artery calcification detected based on a CT image of the lung field in an axial cross section.

FIG. 7 is a flowchart illustrating an example of a processing procedure of diagnosis support processing performed by a diagnosis support system according to the first embodiment.

FIG. 8 is a diagram showing an example of an image interpretation report acquired by the diagnosis support system according to the first embodiment.

FIG. 9 is a diagram showing an example of an image interpretation report to which an evaluation result of a cardiovascular disease risk is added by the diagnosis support system according to the first embodiment.

FIG. 10 is a diagram schematically showing a flow of data when diagnosis support is performed by the diagnosis support system according to the first embodiment.

FIG. 11 is a diagram showing an example of a configuration of a diagnosis support system according to a second embodiment.

FIG. 12 is a flowchart illustrating a processing procedure of diagnosis support processing performed by the diagnosis support system according to the second embodiment.

### DETAILED DESCRIPTION

In general, according to one embodiment, a diagnosis support system evaluates a cardiovascular disease risk by using calcification other than cardiovascular calcification (hereinafter referred to as “extra-coronary calcification (ECC)”) as a trigger. The diagnosis support system includes a processing circuit. The processing circuit is configured to acquire image interpretation information related to an image interpretation result of a photographed image of a patient, determine whether calcification is present or not based on the image interpretation information, evaluate the cardiovascular disease risk based on medical information on a patient, and output a determination result and an evaluation result.

Hereinafter, embodiments of a diagnosis support system will be described in detail with reference to the drawings. In the following description, structural elements having approximately the same function and configuration will be assigned the same reference symbol, and a repeat description will be given only where necessary.

### First Embodiment

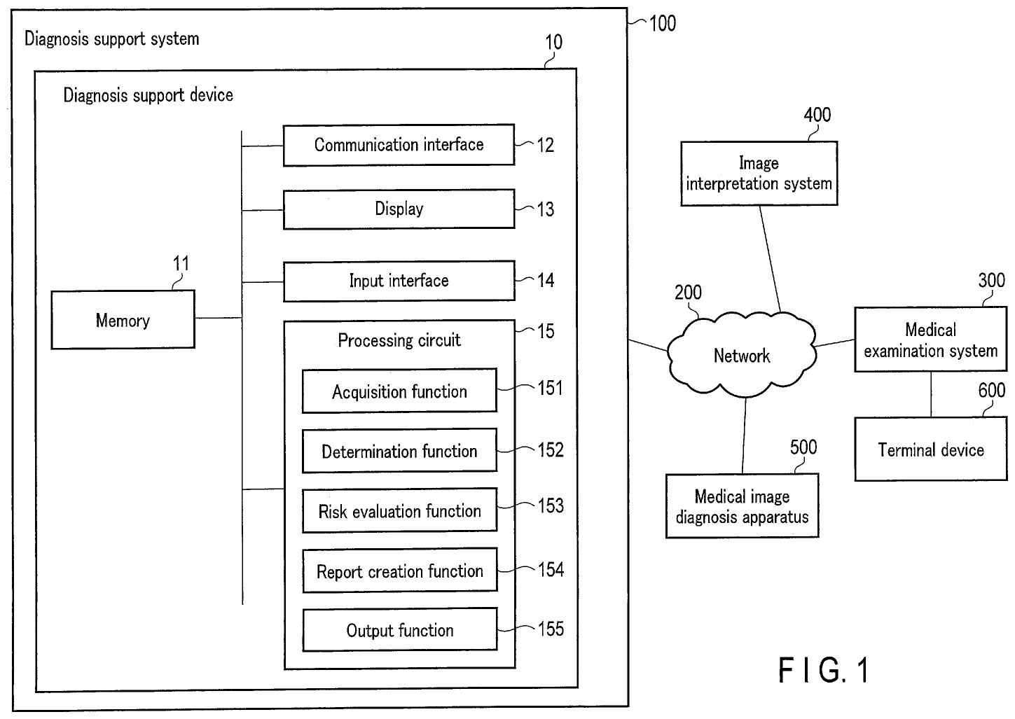

FIG. 1 is a diagram showing a configuration of a diagnosis support system 100. The diagnosis support system 100 is connected to a medical examination system 300, an image interpretation system 400, and a medical image diagnosis apparatus 500 via a network 200.

The network 200 is, for example, a Local Area Network (LAN). The connection to the network 200 may be either wired or wireless. Furthermore, as long as security is ensured by means of a Virtual Private Network (VPN), etc., a line to be connected is not necessarily a LAN. The connection may be made to a public communication line such as the Internet.

The medical examination system 300 manages information related to medical examinations of patients. The medical examination system 300 is, for example, a Hospital Information System (HIS) that manages information related to a medical facility such as a hospital. Under the medical examination system 300, a storage device stores an Electronic Medical Record (EMR), Personal Health Information (PHR), electronic medical records of a subject, information on various examinations, diagnosis results, etc. The medical examination system 300 may also be referred to as a medical information system. A patient may also be referred to as an examinee.

The medical examination system 300 can acquire an interpretation report on a patient from the diagnosis support system 100 via the network 200. Furthermore, the medical examination system 300 is connected to a terminal device 600 via a network. The terminal device 600 is, for example, a PC terminal used by a patient's family doctor or a smartphone used by the patient himself or herself. By transmitting an image interpretation report to the terminal device 600, the medical examination system 300 can present the image interpretation report to the patient's family doctor or the patient himself or herself.

The image interpretation system 400 manages information related to image interpretation of medical images. The image interpretation system 400 is, for example, a medical image management system (such as a Picture Archiving and Communication System: PACS). The image interpretation system 400 stores in the storage device a medical image output from the medical image diagnosis apparatus 500, examination information, an image interpretation report created based on the medical image, etc., in such a manner that they are associated with each other. The image interpretation system 400 includes a display device configured to display a medical image output from the medical image diagnosis apparatus 500, an image interpretation report creating device for an image interpretation doctor who has checked a medical image in order to create an image interpretation report, and so on.

The medical image diagnosis apparatus 500 is, for example, a mammography apparatus, an X-ray computed tomography apparatus, a magnetic resonance imaging apparatus, an ultrasonic diagnosis apparatus, an X-ray diagnosis apparatus, etc.

The diagnosis support system 100 can transmit and receive various types of information to and from the medical examination system 300, the image interpretation system 400, and the medical image diagnosis apparatus 500 via the network 200. The diagnosis support system 100 evaluates the risk of developing cardiovascular disease (CVD) (hereinafter referred to as a cardiovascular disease risk) by using calcification other than cardiovascular calcification (hereinafter referred to as “extra-coronary calcification (ECC)”) as a trigger. Specifically, the diagnosis support system 100 evaluates the cardiovascular disease risk by using, as a trigger, calcification found based on an image (hereinafter referred to as an examination image) acquired by an examination (hereinafter referred to as a non-target examination) other than a heart examination. In other words, the diagnosis support system 100 evaluates the cardiovascular disease risk by using, as a trigger, calcification obtained based on an image through an examination not intended for an examination of the heart. Such an examination image is acquired by an examination order for a non-target examination. Examples of the examination order of the non-target examination include information on examination contents such as an examination site (a site other than the heart), a disease name, an examination purpose, modality, etc., that is, examination information. In addition, the examination order includes information such as an examination ID, an examination date, a patient ID of a patient who is undergoing an examination, a patient name, etc.

Cardiovascular disease is a disease in the circulatory system such as the heart and blood vessels. Cardiovascular disease includes, for example, heart disease and vascular disease.

Calcification found based on an examination image is vascular calcification. Calcification found based on an examination image acquired through a non-target examination may be referred to as incidental findings. Examples of the non-target examination include a mammography examination, a chest X-ray examination, a lung field CT examination, etc. Examples of the examination image include a mammography image, a chest X-ray image, a lung field CT image, etc.

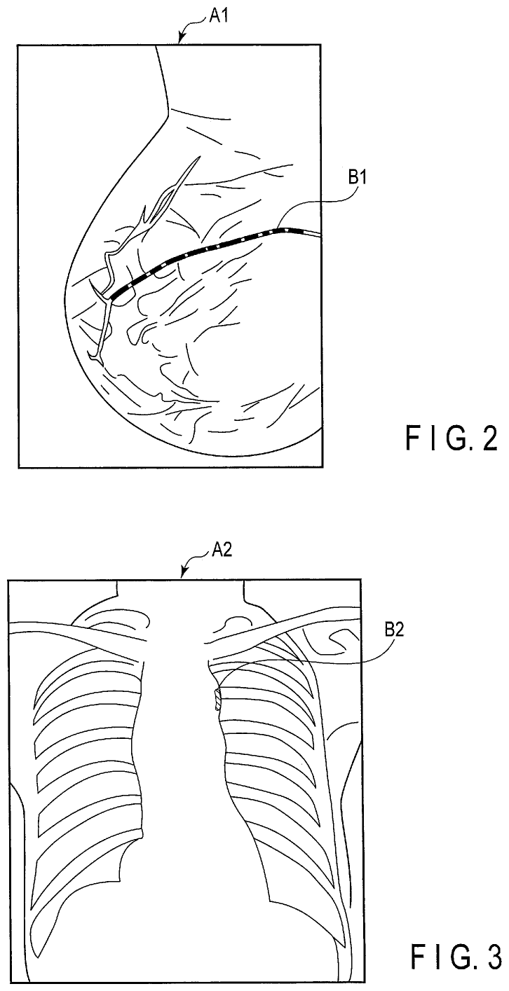

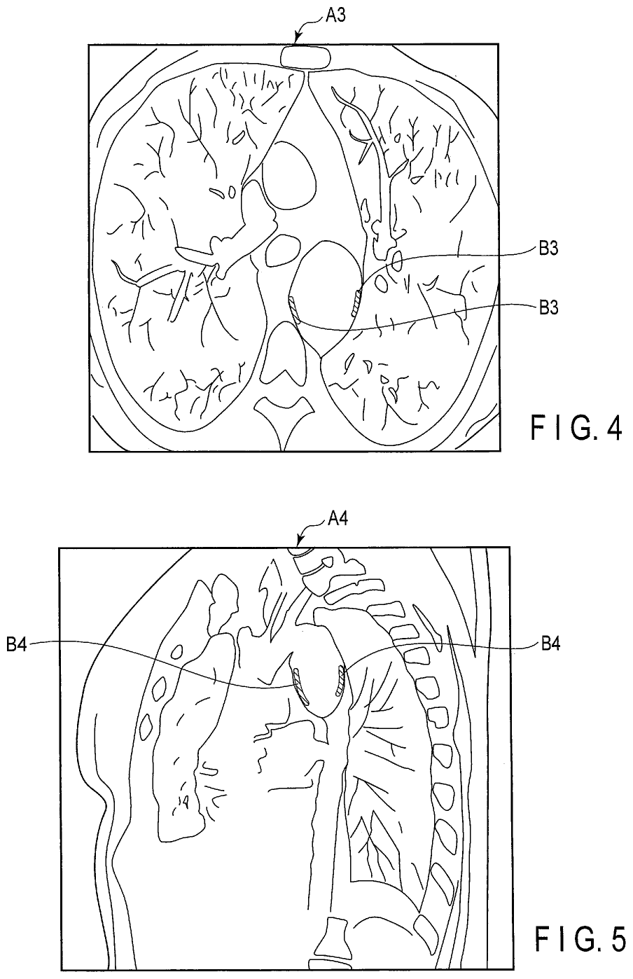

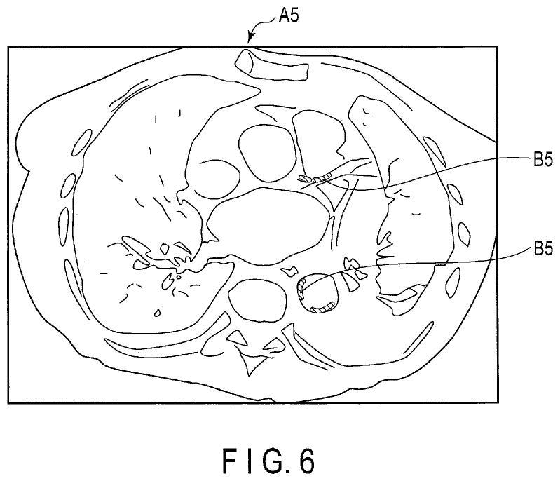

FIG. 2 is a diagram showing an example of breast arterial calcification (BAC) B1 found based on a mammography image A1. FIG. 3 is a diagram showing an example of aortic calcification B2 found based on a chest X-ray image A2. FIG. 4 is a diagram showing an example of aortic calcification B3 found based on a lung field CT image A3 in an axial cross section (body axis cross section). FIG. 5 is a diagram showing an example of aortic calcification B4 found based on a lung field CT image A4 in a sagittal cross section. FIG. 6 is a diagram showing an example of coronary artery calcification B5 found based on a lung field CT image A5 in an axial cross section.

The diagnosis support system 100 includes a diagnosis support device 10. The diagnosis support device 10 includes a memory 11, a communication interface 12, a display 13, an input interface 14, and a processing circuit 15. Hereinafter, the diagnosis support device 10 will be described as a single device executing a plurality of functions; however, a plurality of functions may be respectively executed by different devices. For example, functions executed by the diagnosis support device 10 may be respectively distributed and mounted in different console devices or workstation devices.

The memory 11 is a storage device such as a Hard Disk Drive (HDD), a Solid State Drive (SSD), or an integrated circuit which stores various types of information. Other than the HDD and SSD, the memory 11 may be a portable storage medium such as a Compact Disc (CD), a Digital Versatile Disc (DVD), a flash memory, etc. Alternatively, the memory 11 may be a driver that writes and reads various types of information to and from a semiconductor memory such as a flash memory, a random-access memory (RAM), etc. A storage area of the memory 11 may be located in the diagnosis support device 10 or in an external storage device connected via a network.

The memory 11 stores a program to be executed by the processing circuit 15, various types of data to be used for the processing by the processing circuit 15, etc. Such a program may be installed in advance in a computer from a network or a non-transitory computer-readable storage medium, for example, so that the program will cause the computer to implement the respective functions of the processing circuit 15. Various types of data handled in the disclosure herein are typically digital data. The memory 11 is an example of a storage.

The communication interface 12 is a network interface that controls transmission of communications with the image interpretation system 400, the medical examination system 300, and other external devices via the network 200.

The display 13 displays thereon various types of information. For example, the display 13 outputs medical information generated by the processing circuit 15, a graphical user interface (GUI) for receiving various operations from an operator, etc. For example, the display 13 is a liquid crystal display or a CRT (cathode ray tube) display. The display 13 may display an image interpretation report. The display 13 is an example of a displayer.

The input interface 14 receives various input operations from an operator, converts the received input operations into electric signals, and outputs the converted electric signals to the processing circuit 15. For example, the input interface 14 receives input of medical information, input of various command signals, etc., from the operator. The input interface 14 is implemented by a mouse and keyboard for executing various types of processing of the processing circuit 15, a trackball, a switch button, a touch screen in which a display screen and a touch pad are integrated, a non-contact input circuit using an optical sensor, a voice input circuit, etc. The input interface 14 is connected to the processing circuit 15, converts an input operation received from an operator into an electric signal, and outputs the converted electric signal to the control circuit. In this disclosure, the input interface is not limited to one provided with physical operating components such as a mouse and a keyboard. Examples of the input interface include an electric-signal processing circuit that receives an electric signal corresponding to an input operation from an external input device provided separately from the device, and outputs the received electric signal to the processing circuit 15. The input interface 14 is one example of an inputter.

The processing circuit 15 takes total control over operations of the diagnosis support device 10. The processing circuit 15 is a processor that reads and executes a program in the memory 11, thereby executing an acquisition function 151, a determination function 152, a risk evaluation function 153, a report creation function 154, and an output function 155.

FIG. 1 illustrates an example based on the premise that the acquisition function 151, the determination function 152, the risk evaluation function 153, the report creation function 154, and the output function 155 are realized by the single processing circuit 15; however, the processing circuit may be constituted by a combination of multiple independent processors and the functions are implemented by the processors executing programs, respectively. Furthermore, the acquisition function 151, the determination function 152, the risk evaluation function 153, the report creation function 154, and the output function 155 may also be referred to as an acquisition circuit, a determination circuit, a risk evaluation circuit, a report creation circuit, and an output circuit, respectively, and they may be implemented as separate hardware circuits, respectively. The description made in the above for each function executed by the processing circuit 15 also applies to each embodiment and modification to be described below.

Furthermore, the diagnosis support device 10 will be described based on the premise that a single console executes a plurality of functions; however, a plurality of functions may be executed by individual devices. For example, functions of the processing circuit 15 may be distributed and mounted in different devices.

The term “processor” used herein refers to, for example, a central processing unit (CPU) or a graphics processing unit (GPU), or a circuit such as an application-specific integrated circuit (ASIC), a programmable logic device (such as a simple programmable logic device (SPLD)), a complex programmable logic device (CPLD), a field programmable gate array (FPGA)), etc. In the case of the processor being a CPU, for example, the processor implements a function by reading and executing a program stored in a storage circuit. On the other hand, in the case of the processor being, for example, an ASIC, instead of a program being stored in a storage circuit, a corresponding function is directly incorporated as a logic circuit in the circuit of the processor. Each processor of the present embodiment is not limited to a configuration as a single circuit; a plurality of independent circuits may be combined into one processor to implement the function of the processor. Furthermore, a plurality of components in FIG. 1 may be integrated into one processor to implement their functions. The above description of the “processor” is applicable to the subsequent embodiments and modifications.

The processing circuit 15 acquires, through the acquisition function 151, information related to an image interpretation result of a photographed image of a patient (hereinafter, referred to as image interpretation information). The image interpretation information includes information on extra-coronary calcification. Specifically, the processing circuit 15 acquires, through the acquisition function 151, information on an image interpretation result with respect to an examination image. The processing circuit 15 that implements the acquisition function 151 is an example of the acquirer.

Examples of the image interpretation information include an interpretation report, a diagnosis result of computer-aided diagnosis (hereinafter referred to as CAD), an electronic medical record, etc. In the case of the image interpretation information being an image interpretation report, the processing circuit 15 acquires the image interpretation report from, for example, the image interpretation system 400. In the case of the image interpretation information being a diagnosis result of CAD, the processing circuit 15 acquires the diagnosis result of CAD from, for example, the medical examination system 300 or the image interpretation system 400. Alternatively, the processing circuit 15 may acquire a diagnosis result of CAD by acquiring an examination image from the image interpretation system 400 and executing the CAD on the examination image.

The processing circuit 15 determines, through the determination function 152, whether or not calcification is present based on the image interpretation information. For example, in the case of the image interpretation information being an image interpretation report, the processing circuit 15 determines whether or not vascular calcification is present based on calcification finding information described in the image interpretation report. In the case of the interpretation information being the diagnosis result of CAD, the processing circuit 15 determines whether or not vascular calcification is present based on the diagnosis result of CAD, for example. The processing circuit 15 that implements the determination function 152 is an example of a determiner.

In the case of determining through the risk evaluation function 153 that calcification is present, the processing circuit 15 evaluates the cardiovascular disease risk based on medical information on a patient who has undergone a non-target examination. Specifically, the processing circuit 15 acquires medical information necessary for evaluating the cardiovascular disease risk of a patient, and evaluates the cardiovascular disease risk based on the medical information. At this time, the processing circuit 15 acquires, for example, a PHR of the patient from the medical examination system 300, and extracts medical information necessary for evaluating the cardiovascular disease risk from the acquired PHR. The processing circuit 15 that implements the risk evaluation function 153 is an example of an evaluator.

The evaluation of the cardiovascular disease risk is, for example, a numerical value indicating the risk of developing the cardiovascular disease (hereinafter referred to as a CVD risk value). In this case, if it is determined that calcification is present, the processing circuit 15 acquires medical information necessary for calculating the CVD risk value from the medical examination system 300, and calculates the CVD risk value based on the acquired medical information. Examples of the method of calculating the CVD risk value include a CVD risk calculation tool such as ACC/AHH, Framingham, JBS3, Assign Score, Qrisk2, etc. The CVD risk calculation tool to be used may be preset by a user or may be selected by the user when calculating a CVD risk value. Furthermore, instead of acquiring the medical information necessary for calculating the CVD risk value from the medical examination system 300, the medical information may be input by a user. The medical information necessary for calculating the CVD risk value differs depending on the CVD risk calculation tool. Examples of the medical information necessary for calculating the CVD risk value include age, gender, a total cholesterol level, an HDL cholesterol level, a blood pressure, smoking habits, diabetes (blood sugar level), etc. The CVD risk value may be referred to as a risk value of cardiovascular disease.

In the case of determining through the report creation function 154 that calcification is present, the processing circuit 15 adds an evaluation result to an image interpretation report. Specifically, in the case of determining through the determination function 152 that calcification is present, the processing circuit 15 adds an evaluation result calculated using the risk evaluation function 153 to the image interpretation report on a patient. If the evaluation result is a CVD risk value, the processing circuit 15 adds the CVD risk value to the image interpretation report. The processing circuit 15 that implements the report creation function 154 is an example of a report creator.

The processing circuit 15 outputs, through the output function 155, a determination result of calcification obtained through the determination function 152 and an evaluation result of the cardiovascular disease risk obtained through the risk evaluation function 153. For example, the processing circuit 15 outputs the evaluation result of the cardiovascular disease risk along with the image interpretation information including an image interpretation result related to the presence or absence of calcification. In this case, the processing circuit 15 outputs the evaluation result to the medical examination system 300, the image interpretation system 400, the medical image diagnosis apparatus 500, etc., via the network 200. Furthermore, for example, in the case of the evaluation result being a CVD risk value, the processing circuit 15 outputs an image interpretation report in which the CVD risk value is described. The processing circuit 15 that implements the output function 155 is an example of an outputter.

Next, the operation of diagnosis support processing executed by the diagnosis support system 100 will be described. The diagnosis support processing is processing that acquires image interpretation information on an image interpretation result with respect to an examination image acquired through a non-target examination, determines based on the image interpretation information whether or not calcification is present, evaluates, in the case of determining that calcification is present, the cardiovascular disease risk for a patient who has undergone an examination other than a heart examination, and outputs an evaluation result along with the image interpretation information. The diagnosis support system 100 executes the diagnosis support processing based on the fact that, for example, a new image interpretation is acquired from the image interpretation system 400.

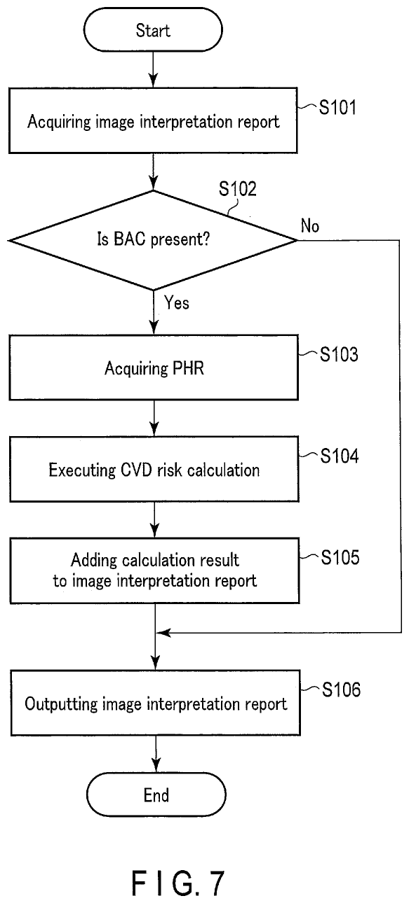

FIG. 7 is a flowchart illustrating an example of a processing procedure of the diagnosis support processing. FIG. 7 illustrates, as one example, the case in which the non-target examination is a “mammography examination”, the examination image is a “mammography image”, the image interpretation information is an “image interpretation report”, and the evaluation result of the cardiovascular disease risk is a “CVD risk value”. Note that the processing procedure described below is only an example, and each processing can be changed as appropriate where possible. Omission, replacement, and addition of a step in the processing procedure described hereinafter can be made as appropriate, in accordance with an actual situation where the present embodiment is realized.

(Diagnosis Support Processing)

(Step S101)

Specifically, the processing circuit 15 acquires, through the acquisition function 151, an image interpretation report related to a mammography image from the image interpretation system 400.

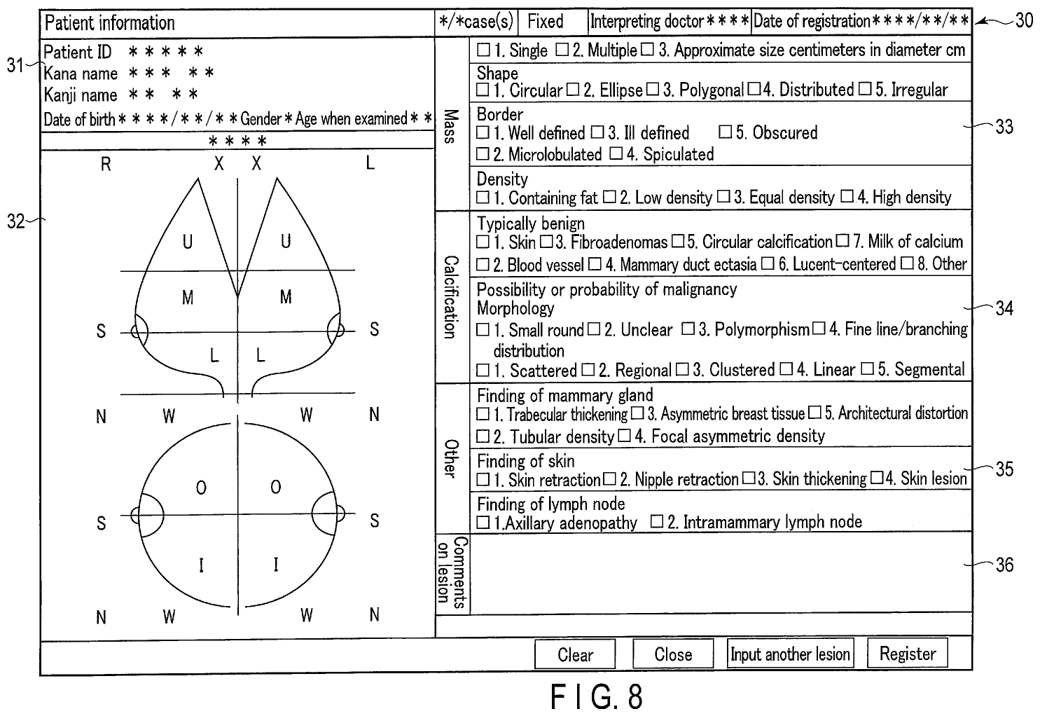

FIG. 8 is a diagram showing an example of the image interpretation report 30 with respect to a mammography image. The image interpretation report 30 shown in FIG. 8 includes a patient information displayer 31, a site information displayer 32, a tumor information displayer 33, a calcification information displayer 34, a finding information displayer 35, and a comment displayer 36. The patient information displayer 31 displays thereon a patient ID, name, date of birth, age, etc. The site information displayer 32 displays thereon information used for designating or selecting a site in the breast. The tumor information displayer 33 displays thereon finding information on a tumor. The tumor information displayer 33 displays thereon, for example, a check box used for selecting a size or shape of a tumor found. The calcification information displayer 34 displays thereon finding information on breast artery calcification. The calcification information displayer 34 displays thereon, for example, a check box used for selecting a feature or morphology of calcification found. The finding information displayer 35 displays thereon, for example, finding information on the mammary gland, the skin, a lymph node, etc. The comment displayer 36 displays thereon an interpreting doctor's comment on a lesion.

(Step S102)

The processing circuit 15 determines, through the determination function 152, whether or not breast artery calcification (BAC) is present, based on calcification finding information described in the calcification information displayer 34 of the image interpretation report 30. In the case of a determination that breast artery calcification is present (step S102-Yes), the processing circuit 15 sequentially executes step S103 and subsequent steps. In the case of a determination that breast artery calcification is not present (step S102-No), the processing proceeds to step S106.

(Step S103)

In the case of determining that breast artery calcification is present, the processing circuit 15 acquires, through the risk evaluation function 153, a PHR of a patient who has undergone a mammography examination from the medical examination system 300.

(Step S104)

The processing circuit 15 extracts, through the risk evaluation function 153, medical information necessary for calculating a CVD risk value from the acquired PHR, and executes calculation of the CVD risk value using the extracted information. In this manner, the CVD risk value of a patient who has undergone a mammography examination is calculated.

(Step S105)

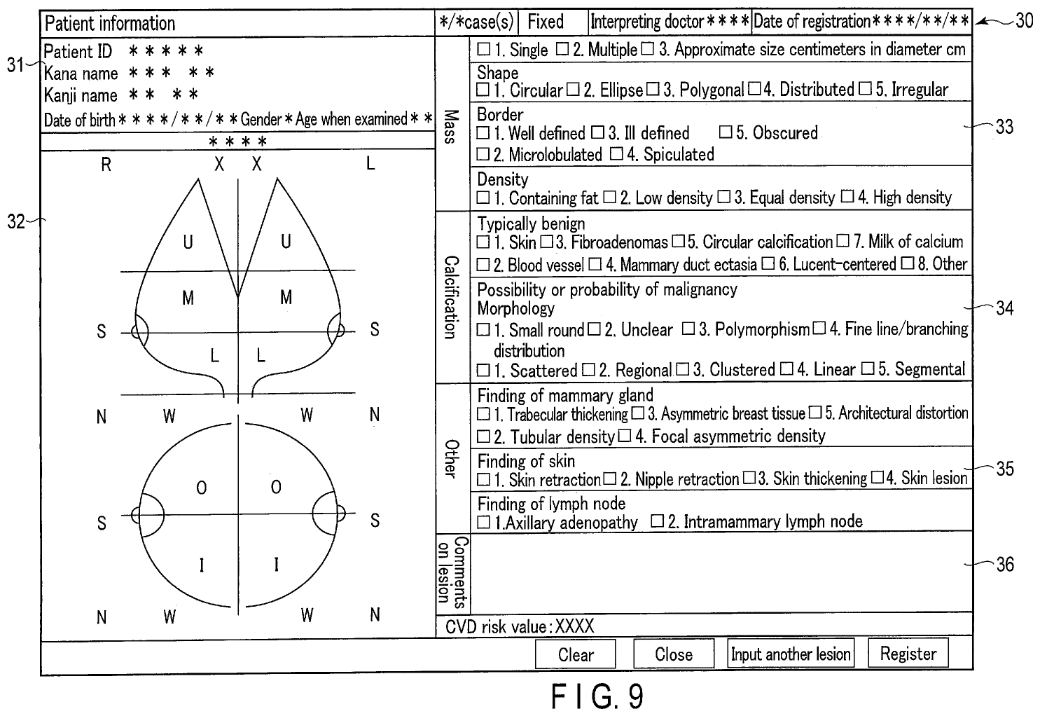

The processing circuit 15 adds, through the report creation function 154, the calculated CVD risk value to an image interpretation report. FIG. 9 is a diagram showing an example in which the CVD risk value is added to the image interpretation report 30 shown in FIG. 8. In the example shown in FIG. 9, the image interpretation report 30 is provided with an evaluation displayer 37. The evaluation displayer 37 displays thereon the calculated CVD risk value.

(Step S106)

The processing circuit 15 outputs, through the output function 155, the image interpretation report which describes the CVD risk value to the medical examination system 300.

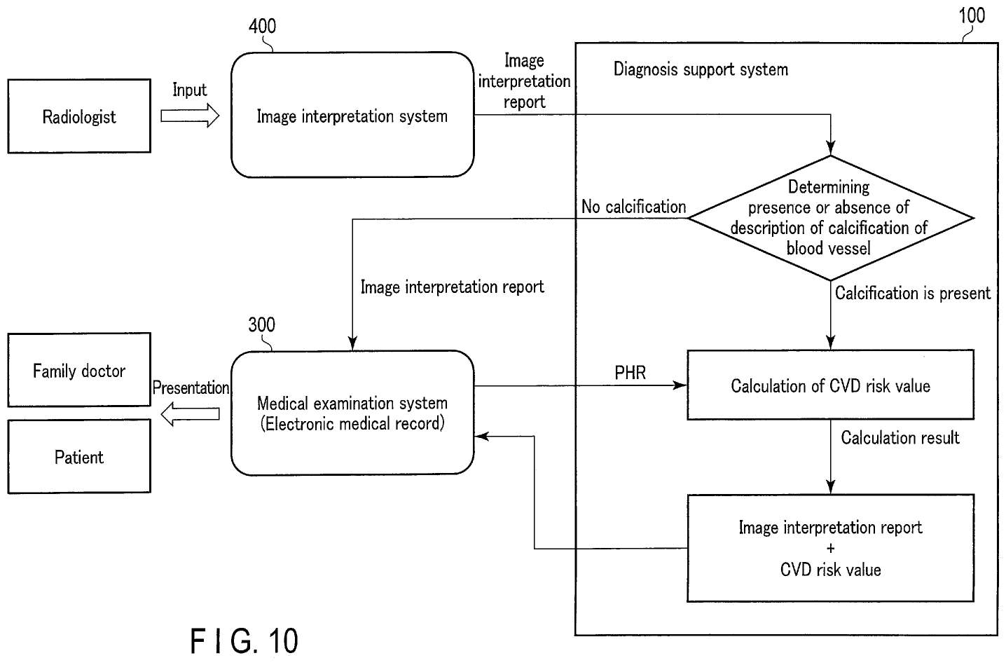

FIG. 10 is a diagram schematically showing a flow of data when diagnosis support is performed by the diagnosis support system 100. As shown in FIG. 10, in the case where diagnosis support is performed by the diagnosis support system 100, first, under the image interpretation system 400, a radiologist inputs an image interpretation result with respect to a mammography image acquired through a mammography examination, so that an image interpretation report with respect to the mammography image is created. The image interpretation system 400 outputs the image interpretation report to the diagnosis support system 100.

As described in the above, the diagnosis support system 100 acquires the interpretation report from the image interpretation system 400, thereby determining whether or not a description indicating that breast artery calcification (BAC) is present is contained in the acquired image interpretation report. In the case of no breast artery calcification, the diagnosis support system 100 outputs the image interpretation report to the medical examination system 300 without adding a description to the image interpretation report. In the case of breast artery calcification being present, the diagnosis support system 100 acquires PHR related to a patient from the medical examination system 300. The diagnosis support system 100 calculates a CVD risk value using the acquired PHR. Thereafter, the diagnosis support system 100 outputs the interpretation report with addition of the CVD value to the medical examination system 300.

The medical examination system 300 acquires the image interpretation report from the diagnosis support system 100. The medical examination system 300 presents the acquired image interpretation report to a medical examination doctor, a patient's family doctor, a patient himself or herself, etc. For example, the medical examination system 300 transmits an e-mail enclosing the image interpretation report to the terminal device 600 used by a patient's family doctor. Alternatively, the medical examination system 300 transmits an email enclosing the image interpretation report to the terminal device 600 such as a smartphone used by the patient himself or herself. In the case of calcification being present in a mammography image, the CVD risk value is described in the image interpretation report.

Hereinafter, the advantageous effects of the diagnosis support system 100 according to the present embodiment will be described.

In recent years, it has been found that vascular calcification other than extra-coronary calcification can be used as a risk marker for cardiovascular disease. For example, it has been found that vascular calcification found from an examination result of a mammography examination or a lung field CT examination can be used as a risk marker for cardiovascular disease. However, in a mammography examination and a lung field CT examination, vascular calcification is only described as a typical benign finding in a report, and no clinical intervention is made in particular. Therefore, in the case where the possibility of a disease risk other than the disease to be examined is indicated from an imaging finding, there is no mechanism for effectively using this information.

The diagnosis support system 100 according to the present embodiment can evaluate the cardiovascular disease risk by using extra-coronary calcification as a trigger. Specifically, the diagnosis support system 100 can evaluate the cardiovascular disease risk by using, as a trigger, calcification found based on an examination image acquired by an examination other than a heart examination (a non-target examination). Calcification is, for example, vascular calcification.

With the above configuration, when a patient undergoes a medical examination that is not intended for a heart examination, such as a lung cancer examination or a breast cancer examination, the diagnosis support system 100 according to the present embodiment can increase interest in another disease such as cardiovascular disease and recommend that the patient undergo an examination for another disease, without increasing the current procedural steps for a radiologist to take. Furthermore, the diagnosis support system 100 according to the present embodiment with the above configuration can contribute to early detection of cardiovascular disease, prevention of cardiovascular disease, and reduction of cardiovascular disease risk.

In addition, it is known that strokes and myocardial infarctions in women increase rapidly after their 50s (after menopause). Furthermore, it is known that cardiovascular disease often develops in women after menopause and the symptoms are atypical. Therefore, there is a tendency for women having cardiovascular disease to take a long time to start treatment and the prognosis tends to be poor. However, in the interpretation of mammographic images, breast artery calcification is generally judged to be “typical benign calcification”. Therefore, even if breast artery calcification is confirmed, no action is taken.

The diagnosis support system 100 according to the present embodiment can be set such that a mammography image is a non-target image, and a mammography image is an examination image. In this case, against the backdrop of Japanese guidelines recommending a mammography examination from the age of 40, the cardiovascular disease risk can be evaluated using a result of the mammography examination that many women regularly undergo at health examinations, etc. This makes it possible to grasp the cardiovascular disease risk at an early stage for cardiovascular disease in women who tend to take a long time to start treatment, thereby leading to the prevention of cardiovascular disease.

It is also known that smokers are two to three times more likely to develop ischemic heart disease or myocardial infarction than nonsmokers.

The diagnosis support system 100 according to the present embodiment can be set such that a lung field CT examination is a non-target examination, and the lung field CT image is an examination image. Alternatively, the diagnosis support system 100 according to the present embodiment can be set such that a chest X-ray examination is a non-target examination, and the chest X-ray image is an examination image. In this case, for example, the cardiovascular disease risk can be evaluated using a result of a lung cancer screening test such as a chest X-ray examination in the non-high-risk group, a low-dose CT examination in the high-risk group, etc. This makes it possible to prevent cardiovascular disease by identifying the risk thereof at an early stage for a patient at high risk of developing ischemic heart disease or myocardial infarction.

Furthermore, the diagnosis support system 100 according to the present embodiment is configured to acquire image interpretation information on an image interpretation result with respect to an examination image, determine based on the image interpretation information whether or not calcification is present, evaluate, in the case of determining that calcification is present, the cardiovascular disease risk for a patient who has undergone a non-target examination, and output an evaluation result along with the image interpretation information. In addition, the diagnosis support system 100 according to the present embodiment can output a determination result as to whether or not calcification is present and an evaluation result of the cardiovascular disease risk.

In the case of the image interpretation information being the image interpretation report, if it is determined that calcification is present, the diagnosis support system 100 can add the evaluation result to the image interpretation report and output the image interpretation report to which the evaluation result is added.

Furthermore, the image interpretation information may be a diagnosis result by computer-aided diagnosis (CAD). In this case, for example, the evaluation result of the cardiovascular disease risk and its meaning are described in a letter for notifying a medical examination doctor, a patient, and a family doctor of the diagnosis result by the CAD.

An interpretation report or letter to which the evaluation result of the cardiovascular disease risk is added is reported to, for example, a medical examination doctor who uses the medical examination system 300. Alternatively, the interpretation report or letter to which the evaluation result of the cardiovascular disease risk is added is transmitted to the terminal device 600, thereby being reported to a family doctor or a patient himself or herself. A medical examination doctor or family doctor can confirm the evaluation result of the cardiovascular disease risk and discuss the next action with a patient. In addition, the patient himself or herself can confirm the evaluation result of the cardiovascular disease risk.

Modification of First Embodiment

The present embodiment describes an example in which the diagnosis support system 100 is mounted as a system separate from the medical examination system 300, the image interpretation system 400, and the medical image diagnosis apparatus 500. However, the diagnosis support system 100 may be mounted on a mammography device of the medical image diagnosis apparatus 500, a display device for an examination image of the image interpretation system 400, or the image interpretation report creating device.

The image interpretation information may be an electronic medical record. In this case, the diagnosis support system 100 acquires the patient's electronic medical record as image interpretation information, and based on the examination result described in the electronic medical record, determines whether or not vascular calcification is present in an examination image obtained through a non-target examination. If vascular calcification is present, the diagnosis support system 100 evaluates the cardiovascular disease risk, adds an evaluation result to the electronic medical record, and outputs the electronic medical record.

In addition, the diagnosis support system 100 may use an examination result from a regular examination to determine whether or not to evaluate the cardiovascular disease risk based on change over time in extra-coronary calcification. For example, even if vascular calcification of blood vessels other than the discovered cardiovascular vessels is small, if the time change of vascular calcification is large, a patient may be judged to be at a high cardiovascular disease risk and thus the cardiovascular disease risk may be evaluated.

In addition to extra-coronary calcification, the proportion of fat in the mammography image may be used to determine whether or not to evaluate the cardiovascular disease risk. For example, a patient's hormonal status may be estimated based on the proportion of fat in a mammography image, and whether or not to evaluate the cardiovascular disease risk may be determined based on an estimation result.

In addition, a determination result by a machine learning model that stratifies the cardiovascular disease risk may be used as the evaluation of the cardiovascular disease risk. In this case, an evaluation result is expressed as, for example, “high risk”, “medium risk”, “low risk”, etc. Instead of calculating a CVD risk value, the diagnosis support system 100 inputs a patient's medical information acquired from the medical examination system 300 into the machine learning model, and outputs a determination result related to the cardiovascular disease risk to the machine learning model. Then, the diagnosis support system 100 adds, as an evaluation of the cardiovascular disease risk, an output result by the machine learning model to an image interpretation report or an electronic medical record, and outputs the report or record. The machine learning model used herein is a trained model that generates a determination result related to the cardiovascular disease risk of a patient based on the medical information of the patient. Furthermore, the machine learning model used herein may be a trained model that accepts input of a medical image such as a CT image or an MRI image of a patient and generates a determination result related to the cardiovascular disease risk of the patient.

### Second Embodiment

Next, the second embodiment will be described. The present embodiment corresponds to the first embodiment modified in a configuration as will be described below. Descriptions of configurations, operations, and advantageous effects similar to those of the first embodiment will be omitted. The diagnosis support system 100 according to the present embodiment generates information for supporting the reduction of the cardiovascular disease risk based on the evaluation result of the cardiovascular disease risk, and proposes this information to a patient.

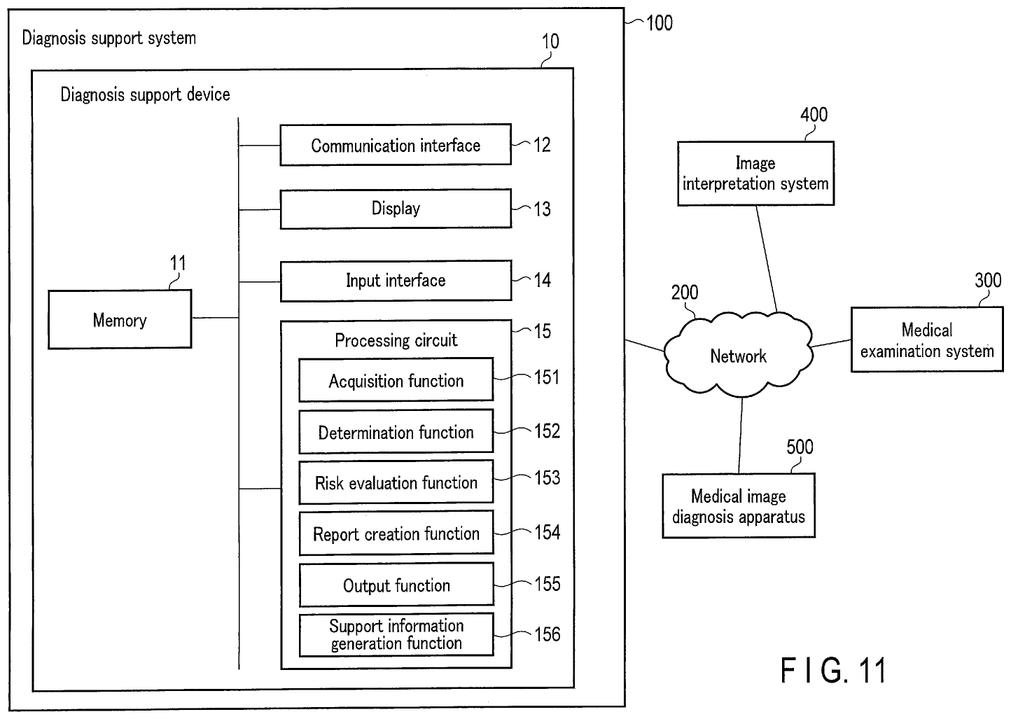

FIG. 11 is a diagram showing a configuration of the diagnosis support system 100 according to the present embodiment. The processing circuit 15 executes a support information generation function 156 in addition to the respective functions described in the first embodiment.

The processing circuit 15 generates, through the support information generation function 156, support information based on an evaluation result of the cardiovascular disease risk. For example, in the case of the cardiovascular disease risk of a patient being high, the processing circuit 15 presents an action for reducing the cardiovascular disease risk according to a patient's situation, according to a CVD risk value. The processing circuit 15 that implements the support information generation function 156 is an example of a support information generator.

The support information is information for encouraging a patient who is judged to have a high cardiovascular disease risk to take an action to reduce the cardiovascular disease risk. Examples of the support information include information on a support application for improving eating habits, a list of cardiologists in the vicinity of the patient's place of residence, information on activities that reduce the cardiovascular disease risk, information that encourages medical examinations, etc. The processing circuit 15 acquires information necessary for generating support information from, for example, a medical examination system 300 or a community medical system, and generates support information based on the acquired information.

The processing circuit 15 outputs, through the output function 155, support information generated through the support information generation function 156 along with the image interpretation information.

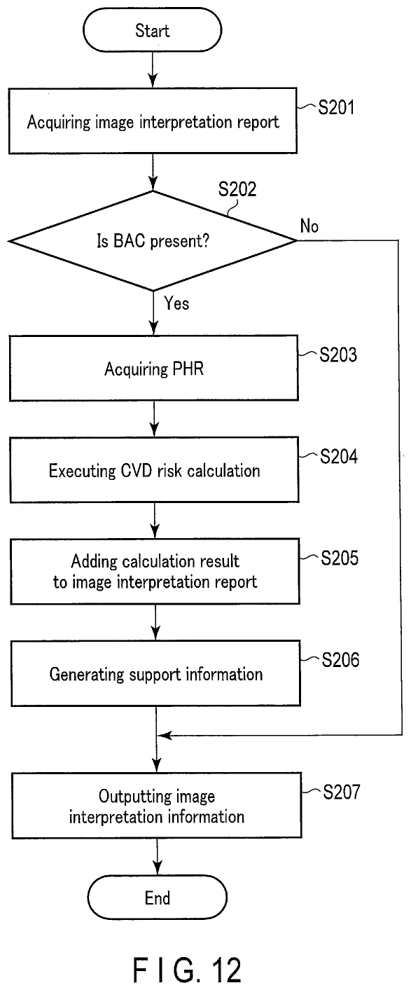

Next, the operation of the diagnosis support processing to be executed by the diagnosis support system 100 according to the present embodiment will be described. FIG. 12 is a flowchart showing an example of a processing procedure of the diagnosis support processing according to the present embodiment. As in FIG. 7, FIG. 12 illustrates the case in which a non-target examination is a “mammography examination”, an examination image is a “mammography image”, image interpretation information is an “image interpretation report”, and a “CVD risk value” is calculated as an evaluation result of the cardiovascular disease risk. The description of the processing from step S201 to step S205 and step S207 will be omitted because it is the same as the processing from step S101 to step S105 in FIG. 7.

(Diagnosis Support Processing)

(Step S206)

The processing circuit 15 generates, through the support information generation function 156, support information based on a calculated CVD risk value.

(Step S207)

The processing circuit 15 outputs, through the output function 155, the generated support information to the medical examination system 300 along with the image interpretation report in which a CVD risk value is described.

The medical examination system 300 presents acquired support information to a medical examination doctor, a patient, and a patient's family doctor. For example, the support information is described in an e-mail for sending a result of a medical examination to a patient.

Hereinafter, the advantageous effects of the diagnosis support system 100 according to the present embodiment will be described.

The diagnosis support system 100 according to the present embodiment generates, through the support information generation function 156, support information based on the evaluation result of the cardiovascular disease risk, and outputs the generated support information along with image interpretation information.

With the above configuration, the diagnosis support system 100 according to the present embodiment can contribute to the prevention of cardiovascular disease and the reduction of the cardiovascular disease risk by presenting an action for reducing the cardiovascular disease risk according to the evaluation result to a patient judged to be at a high cardiovascular disease risk.

Modification of Second Embodiment

A patient to be evaluated in terms of the cardiovascular disease risk may undergo a fecal occult blood examination in addition to a mammography examination at the time of breast cancer screening. In this case, the cardiovascular disease risk estimated based on the patient's intestinal flora may be used as support information.

For example, in the case of determining based on an evaluation result of the cardiovascular disease risk that the cardiovascular disease risk is high, the diagnosis support system 100 issues an additional order using a machine learning algorithm that estimates the cardiovascular disease risk based on the patient's intestinal flora. This machine learning algorithm analyzes the intestinal flora from stool samples, thereby estimating the presence or absence of cardiovascular disease or the long-term risk of developing cardiovascular disease. The diagnosis support system 100 applies an examination result of the fecal occult blood examination and the fecal sample to the aforementioned machine learning algorithm. This can provide, in addition to a calculation result of a CVD risk value based on medical information on a patient, the evaluation result of the cardiovascular disease risk estimated from the intestinal flora of the patient.

In addition, the aforementioned machine learning algorithm may be used as a method for evaluating the cardiovascular disease risk. In this case, in the case of determining that calcification is present in the examination image, the diagnosis support system 100 determines whether or not information on the patient's intestinal flora is recorded. In the case where information on the intestinal flora is recorded, the diagnosis support system 100 assesses the cardiovascular disease risk by applying the information on the patient's intestinal flora to the machine learning algorithm.

According to at least one embodiment described in the above, it is possible to contribute to the reduction of the cardiovascular disease risk.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A diagnosis support system configured to evaluate a cardiovascular disease risk by using extra-coronary calcification as a trigger, the diagnosis support system comprising:
a processing circuit,
wherein the processing circuit is configured to:
acquire image interpretation information related to an image interpretation result of an image of a patient;
determine based on the image interpretation information whether or not the calcification is present;
evaluate the cardiovascular disease risk based on medical information on the patient, and
output a result of said determining and a result of said evaluating.

2. The diagnosis support system according to claim 1, wherein in a case where it is determined that the calcification is present, the processing circuit is configured to evaluate the cardiovascular disease risk based on the medical information on the patient.

3. The diagnosis support system according to claim 1, wherein the processing circuit is configured to calculate a value indicating a risk of developing a cardiovascular disease as an evaluation of the cardiovascular disease risk, and
the medical information includes age, gender, a total cholesterol level, an HDL cholesterol level, a blood pressure, a presence or absence of a smoking habit, and a blood sugar level.

4. A diagnosis support system comprising a processing circuit configured to evaluate a cardiovascular disease risk by using, as a trigger, calcification found based on an image acquired through an examination other than a heart examination.

5. The diagnosis support system according to claim 4, wherein the calcification is vascular calcification.

6. The diagnosis support system according to claim 4, wherein the examination other than a heart examination is a mammography examination, and
the image is a mammography image.

7. The diagnosis support system according to claim 4, wherein the examination other than a heart examination is a lung field CT examination, and
the image is a lung field CT image.

8. The diagnosis support system according to claim 4, wherein the examination other than a heart examination is a chest X-ray examination, and
the image is a chest X-ray image.

9. The diagnosis support system according to claim 4, wherein the processing circuit is configured to:
acquire image interpretation information related to an image interpretation result with respect to the image;
determine whether the calcification is present or not based on the image interpretation information;
in a case where it is determined that the calcification is present, evaluate the cardiovascular disease risk based on medical information on a patient who has undergone the examination other than a heart examination; and
output an evaluation result along with the image interpretation information.

10. The diagnosis support system according to claim 9, wherein the image interpretation information is an image interpretation report.

11. The diagnosis support system according to claim 10, wherein the processing circuit is configured to:
add the evaluation result to the image interpretation report in a case where it is determined that the calcification is present; and
output the image interpretation report to which the evaluation result is added.

12. The diagnosis support system according to claim 9, wherein the image interpretation information is a diagnosis result by computer-aided diagnosis.

13. The diagnosis support system according to claim 9, wherein the processing circuit is configured to:
generate support information based on the evaluation result; and
output the support information along with the image interpretation information.
